(19) United States
(12) Patent Application Publication
(10) Pub. No.: US 2020/0265930 A1
(43) Pub. Date: Aug. 20, 2020

(54) MEDICAL EXAMINATION APPARATUS AND MEDICAL EXAMINATION SYSTEM

(71) Applicant: KONICA MINOLTA, INC., Tokyo (JP)

(72) Inventor: Yusuke TANABE, Tokyo (JP)

(73) Assignee: KONICA MINOLTA, INC., Tokyo (JP)

(21) Appl. No.: 16/785,516

(22) Filed: Feb. 7, 2020

(30) Foreign Application Priority Data
Feb. 15, 2019 (JP) 2019-025044

(51) Int. Cl. G16H 10/60; A61B 5/00; G16H 30/20; G16H 40/63; G16H 40/20; G16H 50/20

(52) U.S. Cl. CPC G16H 10/60; A61B 5/7435; A61B 5/0013; G16H 50/20; G16H 30/20; G16H 40/63; G16H 40/20; A61B 5/0033

(57) ABSTRACT

A medical examination apparatus includes: an examination data generator that generates examination data by examining a subject; a communicator that communicates with a plurality of information processing apparatuses; and a hardware processor that sets an information processing apparatus as an acquisition destination and that, when subject information and apparatus identification information for identifying any one of the plurality of information processing apparatuses are received from the information processing apparatus through the communicator, determines whether the information processing apparatus as a transmission source of the received subject information and apparatus identification information is the information processing apparatus as the acquisition destination based on the received apparatus identification information, and that sets the received subject information as subject information regarding a subject to be examined by the examination data generator when it is determined that the information processing apparatus as the transmission source is the information processing apparatus as the acquisition destination.

### CROSS-REFERENCE TO RELATED APPLICATIONS

The entire disclosure of Japanese Patent Application No. 2019-025044 filed on Feb. 15, 2019 is incorporated herein by reference in its entirety.

### BACKGROUND

### Technological Field

The present invention relates to a medical examination apparatus and a medical examination system.

### Description of the Related Art

When performing an examination in a medical examination apparatus such as an ultrasound diagnostic apparatus, examination data generated by the medical examination apparatus and subject information (subject ID (identification), name, date of birth, sex, and the like) regarding a subject need to be associated with each other. For this reason, instead of inputting subject information on the medical examination apparatus, a method of acquiring subject information from an information processing apparatus, such as an electronic medical record terminal, and starting an examination is used.

For example, a technique has been proposed in which a medical examination apparatus capable of communicating with an information processing apparatus (external apparatus) acquires subject information from the information processing apparatus at a timing when a predetermined operation input is performed (refer to JP 2018-29786A). The technique disclosed in JP 2018-29786A is based on the assumption that one information processing apparatus and one medical examination apparatus are installed.

However, in the actual field, a plurality of information processing apparatuses and a plurality of medical examination apparatuses are installed in many cases. In the case of a hospital having two examination rooms, an information processing apparatus (electronic medical record terminal) is usually installed in each examination room. In a hospital where a plurality of medical examination apparatuses are used, a medical examination apparatus may be installed in each examination room as in the case of the information processing apparatus. However, a medical examination apparatus such as a small ultrasound diagnostic apparatus with high portability is often moved between examination rooms and used.

Thus, an information processing apparatus as a connection destination from which a medical examination apparatus should acquire subject information changes depending on the situation. It is fatal to require setting every time in order to change the connection destination in the emergency medical field.

Accordingly, there is a need for a method allowing each medical examination apparatus to acquire subject information from a desired information processing apparatus in a situation in which there are a plurality of medical examination apparatuses and a plurality of information processing apparatuses.

A technique has been proposed in which a multi-purpose electronic display device such as a smartphone acquires selection information of a plurality of ultrasound imaging apparatuses using a first wireless communication protocol (for example, Bluetooth (registered trademark)), selects a desired ultrasound imaging apparatus among the pieces of selection information, establishes a connection with the selected ultrasound imaging apparatus using a second wireless communication protocol (for example, Wi-Fi), and acquires an ultrasound image from the established ultrasound imaging apparatus and displays the ultrasound image (refer to JP 2018-509269A). By using this method, the connection destination can be changed in a situation in which there are a plurality of ultrasound diagnostic apparatuses (corresponding to the above-described “information processing apparatus” in terms of apparatuses on the data providing side) and a plurality of multi-purpose electronic display devices (corresponding to the above-described “medical examination apparatus” in terms of apparatuses on the data acquisition side).

### SUMMARY

However, in the method using two wireless communication protocols as in the related art, there is a problem that a time is required since it is necessary to make a preliminary connection using another wireless communication protocol (such as Bluetooth) before establishing a connection between apparatuses using Wi-Fi or the like. When communication for preliminary connection is unstable, more time is required.

In addition, since both apparatuses require a hardware configuration, such as Bluetooth, the usage is limited. A general electronic medical record terminal (information processing apparatus) has a Wi-Fi communication function, but the electronic medical record terminal does not necessarily have a wireless communication protocol, such as Bluetooth.

For these reasons, when a medical examination apparatus acquires subject information from an information processing apparatus, a method that can be used in a wider range of situations and that has improved convenience is required.

The present invention has been made in view of the aforementioned problems in the related art, and it is an object of the invention to enable a medical examination apparatus to easily acquire subject information from a desired information processing apparatus even in a situation in which there are a plurality of medical examination apparatuses and a plurality of information processing apparatuses.

To achieve at least one of the abovementioned objects, according to an aspect of the present invention, a medical examination apparatus reflecting one aspect of the present invention comprises: an examination data generator that generates examination data by examining a subject; a communicator that is able to communicate with a plurality of information processing apparatuses; and a hardware processor that sets an information processing apparatus as an acquisition destination among the plurality of information processing apparatuses and that, when subject information regarding a subject and apparatus identification information for identifying any one of the plurality of information processing apparatuses are received from the information processing apparatus through the communicator, determines whether or not the information processing apparatus as a transmission source of the received subject information and apparatus identification information is the information processing apparatus as the acquisition destination based on the received apparatus identification information, and that sets the received subject information as subject information regarding a subject to be examined by the examination data generator when it is determined that the information processing apparatus as the transmission source is the information processing apparatus as the acquisition destination.

To achieve at least one of the abovementioned objects, according to another aspect of the present invention, a medical examination system reflecting one aspect of the present invention comprises: a plurality of information processing apparatuses that input and display subject information regarding a subject; a plurality of medical examination apparatuses each including an examination data generator that generates examination data by examining a subject; and a server that is able to communicate with the plurality of medical examination apparatuses.

Each of the plurality of information processing apparatuses includes a transmitter that transmits subject information displayed on the information processing apparatus and apparatus identification information for identifying the information processing apparatus to each of the plurality of medical examination apparatuses. Each of the plurality of medical examination apparatuses includes: a communicator that is able to communicate with the plurality of information processing apparatuses; and a first hardware processor that sets an information processing apparatus as an acquisition destination among the plurality of information processing apparatuses and that, when subject information regarding a subject and apparatus identification information for identifying any one of the plurality of information processing apparatuses are received from the information processing apparatus through the communicator, determines whether or not the information processing apparatus as a transmission source of the received subject information and apparatus identification information is the information processing apparatus as the acquisition destination based on the received apparatus identification information, and that sets the received subject information as subject information regarding a subject to be examined by the examination data generator when it is determined that the information processing apparatus as the transmission source is the information processing apparatus as the acquisition destination. The server includes a storage that stores a setting table in which each of the plurality of medical examination apparatuses is associated with each of the plurality of information processing apparatuses. The first hardware processor determines whether or not the information processing apparatus as the transmission source is the information processing apparatus as the acquisition destination based on information regarding the setting table acquired from the server.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a system configuration diagram of a medical examination system according to a first embodiment of the present invention;

FIG. 2 is a block diagram illustrating a functional configuration of an electronic medical record terminal;

FIG. 3 is a block diagram illustrating a functional configuration of an ultrasound diagnostic apparatus;

FIG. 4 is a flowchart illustrating a first subject information setting process;

FIG. 5 is a diagram for describing a specific example of the first subject information setting process;

FIG. 6 is a system configuration diagram of a medical examination system according to a second embodiment of the present invention;

FIG. 7 is a block diagram illustrating a functional configuration of a server;

FIG. 8 is a flowchart illustrating a second subject information setting process;

FIG. 9 is a diagram for describing a specific example of the second subject information setting process;

FIG. 10 is a flowchart illustrating an acquisition destination change mode process in a third embodiment of the present invention;

FIG. 11 is a diagram for describing a specific example of the acquisition destination change mode process;

FIG. 12A is a specific example of a setting table;

FIG. 12B is a specific example of a setting table;

FIG. 12C is a specific example of a setting table;

FIG. 13 is a flowchart illustrating a setting table correction process in a fourth embodiment of the present invention;

FIG. 14 is an example of a notification screen displayed on an ultrasound diagnostic apparatus;

FIG. 15A is a specific example of a setting table;

FIG. 15B is a specific example of a setting table;

FIG. 15C is a specific example of a setting table;

FIG. 16 is an example of a setting screen displayed on an ultrasound diagnostic apparatus;

FIG. 17 is a flowchart illustrating a third subject information setting process in a fifth embodiment of the present invention; and

FIG. 18 is an example of a warning screen displayed on an ultrasound diagnostic apparatus.

### DETAILED DESCRIPTION OF THE EMBODIMENTS

Hereinafter, a medical examination apparatus and a medical examination system according to embodiments of the present invention will be described with reference to the accompanying diagrams. However, the scope of the invention is not limited to the disclosed embodiments. In addition, the scope of the invention is not limited to the illustrated examples.

### First Embodiment

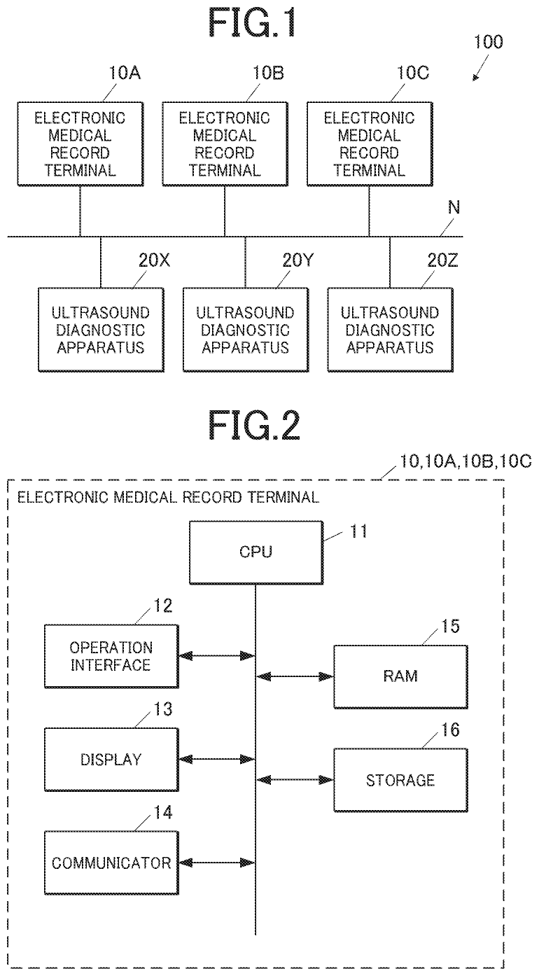

First, a first embodiment of the present invention will be described. FIG. 1 illustrates a system configuration of a medical examination system 100 according to the first embodiment. As illustrated in FIG. 1, the medical examination system 100 is configured to include electronic medical record terminals 10A, 10B, and 10C as a plurality of information processing apparatuses and ultrasound diagnostic apparatuses 20X, 20Y, and 20Z as a plurality of medical examination apparatuses. The electronic medical record terminals 10A, 10B and 10C and the ultrasound diagnostic apparatuses 20X, 20Y, and 20Z are connected to each other through a communication network N, such as a local area network (LAN), so that data communication is possible. The number of electronic medical record terminals 10A, 10B, and 10C and ultrasound diagnostic apparatuses 20X, 20Y, and 20Z is not particularly limited. The medical examination system 100 is installed in a hospital. Each apparatus configuring the medical examination system 100 conforms to the digital image and communications in medicine (DICOM) standard, and communication between apparatuses is performed according to the DICOM.

The electronic medical record terminals 10A, 10B, and 10C are installed in an examination room where a doctor examines and treats a patient, and is used when inputting and displaying the electronic medical record. The electronic medical record includes subject information regarding a subject (patient). The subject information includes a subject ID for identifying the subject, a name (kanji, kana, and romaji) of the subject, date of birth, sex, and the like. Hereinafter, the electronic medical record terminals 10A, 10B, and 10C may be referred to as an electronic medical record terminal 10.

A doctor performs an examination on a subject (patient) using the ultrasound diagnostic apparatuses 20X, 20Y, and 20Z as necessary. Hereinafter, the ultrasound diagnostic apparatuses 20X, 20Y, and 20Z may be referred to as an ultrasound diagnostic apparatus 20.

The ultrasound diagnostic apparatus 20 is a medical examination apparatus that displays and outputs an ultrasound image of a state of a living body tissue of a subject. The ultrasound diagnostic apparatus 20 transmits an ultrasound wave (transmission ultrasound wave) to the inside of the subject and receives a reflected wave (reflected ultrasound wave: echo) of the ultrasound wave reflected inside the subject. The ultrasound diagnostic apparatus 20 converts the received reflected ultrasound wave into an electrical signal, and generates ultrasound image data (examination data) based on the electrical signal. The ultrasound diagnostic apparatus 20 displays the internal state in the subject as an ultrasound image based on the generated ultrasound image data. The ultrasound diagnostic apparatus 20 generates supplementary information regarding the generated ultrasound image data based on the subject information and the like. The ultrasound diagnostic apparatus 20 adds the supplementary information to the ultrasound image data to generate an image file configured to include DICOM image data conforming to the DICOM standard.

FIG. 2 illustrates a functional configuration of the electronic medical record terminal 10. As illustrated in FIG. 2, the electronic medical record terminal 10 includes a central processing unit (CPU) 11, an operation interface 12, a display 13, a communicator 14, a random access memory (RAM) 15, and a storage 16.

The CPU 11 reads various programs such as system programs and processing programs stored in the storage 16 and loads the programs to the RAM 15, and controls the operation of each unit of the electronic medical record terminal 10 according to the loaded programs.

The operation interface 12 includes a keyboard having cursor keys, character and number input keys, various function keys, and the like, and a pointing device such as a mouse. A press signal of a key pressed on the keyboard and a position input signal by the pointing device are output to the CPU 11 as operation signals. For example, the operation interface 12 is used when inputting subject information.

The display 13 is configured to include a monitor such as a liquid crystal display (LCD), and displays various screens according to instructions of a display signal input from the CPU 11. For example, the display 13 displays an electronic medical record including subject information.

The communicator 14 includes a network interface and the like, and transmits and receives data to and from an external apparatus such as the ultrasound diagnostic apparatus 20 connected through the communication network N.

The RAM 15 is a volatile semiconductor memory, and forms a work area where various programs that can be executed by the CPU 11, input or output data, parameters, and the like, which are read from the storage 16, are temporarily stored in various kinds of processing executed by the CPU 11.

The storage 16 is configured by a hard disk drive (HDD), a nonvolatile semiconductor memory, or the like, and stores a system program or various programs executed by the CPU 11.

The storage 16 stores subject information corresponding to a plurality of subjects (patients). The storage 16 stores an apparatus ID (identification) as apparatus identification information for identifying the electronic medical record terminal 10. The apparatus ID is a serial number, an IP address, or the like of the electronic medical record terminal 10.

The CPU 11 causes each of the plurality of ultrasound diagnostic apparatuses 20 to transmit subject information displayed on the electronic medical record terminal 10 and the apparatus ID for identifying the electronic medical record terminal 10 through the communicator 14 (transmitter). The subject information transmitted from the electronic medical record terminal 10 to the ultrasound diagnostic apparatus 20 includes a subject ID.

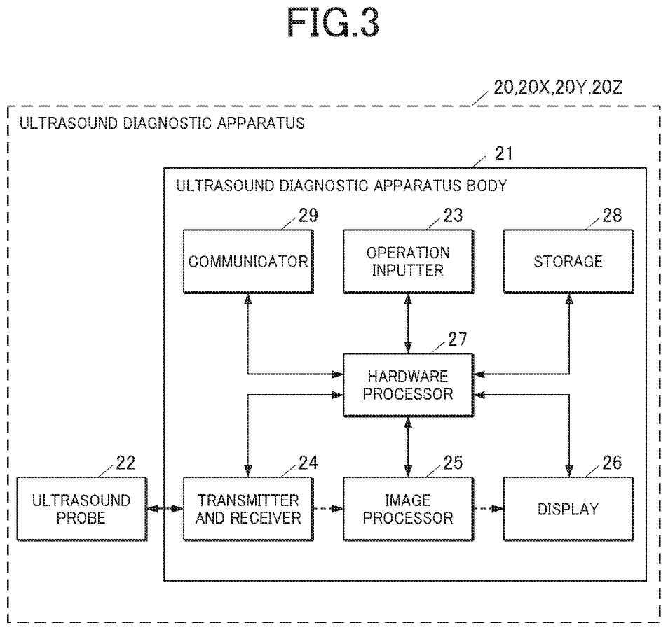

FIG. 3 illustrates a functional configuration of the ultrasound diagnostic apparatus 20. As illustrated in FIG. 3, the ultrasound diagnostic apparatus 20 includes an ultrasound diagnostic apparatus body 21 and an ultrasound probe 22. The ultrasound probe 22 transmits a transmission ultrasound wave to the inside of the subject and receives a reflected ultrasound wave from the inside of the subject. The ultrasound diagnostic apparatus body 21 is connected to the ultrasound probe 22 through a cable, and transmits a driving signal as an electrical signal to the ultrasound probe 22 so that the ultrasound probe 22 transmits a transmission ultrasound wave to the inside of the subject. The ultrasound diagnostic apparatus body 21 receives a reception signal that is an electrical signal generated by the ultrasound probe 22 in accordance with the reflected ultrasound wave from the inside of the subject received by the ultrasound probe 22, and generates ultrasound image data.

The ultrasound diagnostic apparatus body 21 includes an operation inputter 23, a transmitter and receiver 24, an image processor 25, a display 26, a hardware processor 27 (hardware processor, first hardware processor), a storage 28, and a communicator 29.

The operation inputter 23 includes various switches, buttons, a trackball, a mouse, a keyboard, and the like for inputting data, such as subject information, and various parameters for displaying an ultrasound image on the display 26, and outputs an operation signal to the hardware processor 27.

The transmitter and receiver 24 is a circuit that supplies a driving signal, which is an electrical signal, to the ultrasound probe 22 so that the ultrasound probe 22 generates a transmission ultrasound wave and receives a reception signal, which is an electrical signal, from the ultrasound probe 22 under the control of the hardware processor 27. The transmitter and receiver 24 performs a scan by driving a plurality of transducers arrayed in the ultrasound probe 22 to generate transmission ultrasound waves. The transmitter and receiver 24 amplifies a reception signal at a predetermined gain and performs A/D conversion, and then performs reception beamforming by performing phasing addition processing.

The image processor 25 performs envelope detection processing, logarithmic compression, and the like on the reception signal subjected to the phasing addition processing and adjusts the dynamic range or the gain to perform brightness conversion, thereby generating B-mode image data. In the B-mode image data, the strength of the received signal is expressed by brightness. The image processor 25 generates ultrasound image data in units of frames from the B-mode image data generated as described above. The ultrasound image data in units of frames may be referred to as frame image data. The image processor 25 includes, for example, a large-capacity memory that can hold 10-second frame image data.

The transmitter and receiver 24 and the image processor 25 configure an examination data generator that examines a subject and generates examination data (ultrasound image data).

As the display 26, a display device such as an LCD can be applied. The display 26 displays an ultrasound image based on the frame image data generated by the image processor 25 on the display screen.

The hardware processor 27 is configured to include a CPU, a read only memory (ROM), and a RAM, and reads various processing programs such as a system program stored in the ROM, loads the processing program to the RAM, and controls the operation of each unit of the ultrasound diagnostic apparatus 20 according to the loaded program. The RAM forms a work area for temporarily storing various programs executed by the CPU and data related to these programs. The ROM is configured by a nonvolatile semiconductor memory or the like, and stores a system program for the ultrasound diagnostic apparatus 20, various processing programs that can be executed on the system program, various kinds of data, and the like. These programs are stored in the form of computer-readable program code, and the CPU sequentially executes operations according to the program code.

The storage 28 is configured by a large-capacity recording medium, such as an HDD, and stores ultrasound image data generated by the ultrasound diagnostic apparatus 20. The storage 28 can store, as ultrasound image data, still image data that is image data of a still image of one frame and moving image data that is generated so that still image data of several frames can be displayed as a moving image. In addition to the HDD, the storage 28 may include a portable recording medium, such as a compact disk-recordable (CD-R), and a data reader and writer such as a CD-R drive for recording data on the recording medium. The storage 28 may store an image file configured to include DICOM image data.

The storage 28 stores a setting table indicating an association as to whether or not each of the plurality of electronic medical record terminals 10 is an electronic medical record terminal as an acquisition destination. In the first embodiment, the setting table includes information indicating whether or not each of the plurality of electronic medical record terminals 10 is an “electronic medical record terminal as an acquisition destination” for the host apparatus (ultrasound diagnostic apparatus 20), and accordingly, the content of the setting table differs depending on each ultrasound diagnostic apparatus 20.

The communicator 29 is configured by a network interface or the like, and transmits and receives data to and from an external apparatus such as the electronic medical record terminal 10 connected through the communication network N.

For each unit provided in the ultrasound diagnostic apparatus 20, functions of some or all of the functional blocks can be realized as a hardware circuit, such as an integrated circuit. Alternatively, the functions of some or all of the functional blocks may be executed by software. In this case, the software is stored in one or more storage media such as a ROM, an optical disk, and a hard disk, and the software is executed by the arithmetic processor.

The hardware processor 27 sets an “electronic medical record terminal as an acquisition destination” for the host apparatus, among the plurality of electronic medical record terminals 10, according to an operation through the operation inputter 23. The hardware processor 27 sets an “electronic medical record terminal as an acquisition destination” by changing the setting table stored in the storage 28.

When subject information regarding a subject and an apparatus ID for identifying the electronic medical record terminal 10 are received from any one of the plurality of electronic medical record terminals 10 through the communicator 29, the hardware processor 27 determines whether or not the electronic medical record terminal 10 that has transmitted the received subject information and apparatus ID is an “electronic medical record terminal as an acquisition destination” based on the received apparatus ID.

Specifically, the hardware processor 27 determines whether or not the electronic medical record terminal 10 that has transmitted the subject information and the apparatus ID is an “electronic medical record terminal as an acquisition destination” with reference to the setting table stored in the storage 28.

When it is determined that the electronic medical record terminal 10 that has transmitted the subject information and the apparatus ID is an “electronic medical record terminal as an acquisition destination”, the hardware processor 27 sets the received subject information as subject information regarding a subject to be examined by the ultrasound diagnostic apparatus 20 (examination data generator).

Next, the operation of the first embodiment will be described.

First, the user displays subject information of a subject to be examined on the display 13 in any one of the plurality of electronic medical record terminals 10. In this state, when the user gives an instruction to start an examination by an operation through the operation interface 12, the CPU 11 of the electronic medical record terminal 10 being operated transmits the subject information displayed on the display 13 and the apparatus ID for identifying the electronic medical record terminal 10 to all the ultrasound diagnostic apparatuses 20 in the medical examination system 100 through the communicator 14.

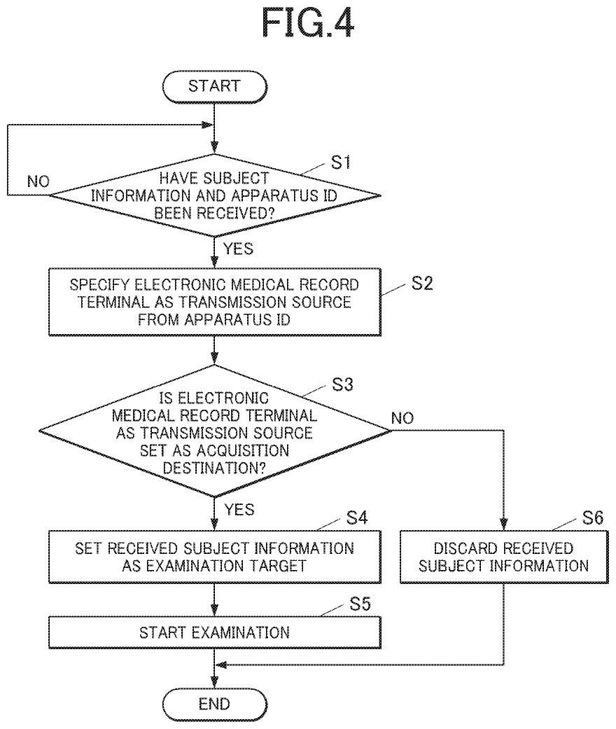

FIG. 4 is a flowchart illustrating a first subject information setting process executed by each ultrasound diagnostic apparatus 20. This process is realized by software processing in cooperation with the CPU of the hardware processor 27 and the program stored in the ROM.

The hardware processor 27 determines whether or not subject information and an apparatus ID have been received from any one of the plurality of electronic medical record terminals 10 through the communicator 29 (step S1). When it is determined that the subject information and the apparatus ID have not been received (step S1; NO), the process returns to step S1.

When it is determined that the subject information and the apparatus ID have been received from any one of the electronic medical record terminals 10 in step S1 (step S1; YES), the hardware processor 27 specifies the electronic medical record terminal 10, which has transmitted the subject information and the apparatus ID, from the received apparatus ID (step S2).

Then, the hardware processor 27 determines whether or not the electronic medical record terminal 10 as a transmission source is set as an “electronic medical record terminal as an acquisition destination” with reference to the setting table stored in the storage 28 (step S3). In the setting table, for example, “0” is assigned to each electronic medical record terminal 10 when the electronic medical record terminal 10 is an “information processing apparatus as an acquisition destination” of the ultrasound diagnostic apparatus 20 having the setting table, and “X” is assigned to each electronic medical record terminal 10 when the electronic medical record terminal 10 is not an “information processing apparatus as an acquisition destination” of the ultrasound diagnostic apparatus 20 having the setting table.

When the electronic medical record terminal 10 as a transmission source is set as an “electronic medical record terminal as an acquisition destination” (step S3; YES), the hardware processor 27 sets the received subject information as subject information regarding a subject to be examined (step S4), and starts an examination (step S5).

The hardware processor 27 stores examination data (ultrasound image data) generated by the ultrasound diagnostic apparatus 20 in the storage 28 so as to be associated with the set subject information.

When the electronic medical record terminal 10 as a transmission source is not set as an “electronic medical record terminal as an acquisition destination” in step S3 (step S3; NO), the hardware processor 27 discards the received subject information (step S6).

After step S5 or step S6, the first subject information setting process ends.

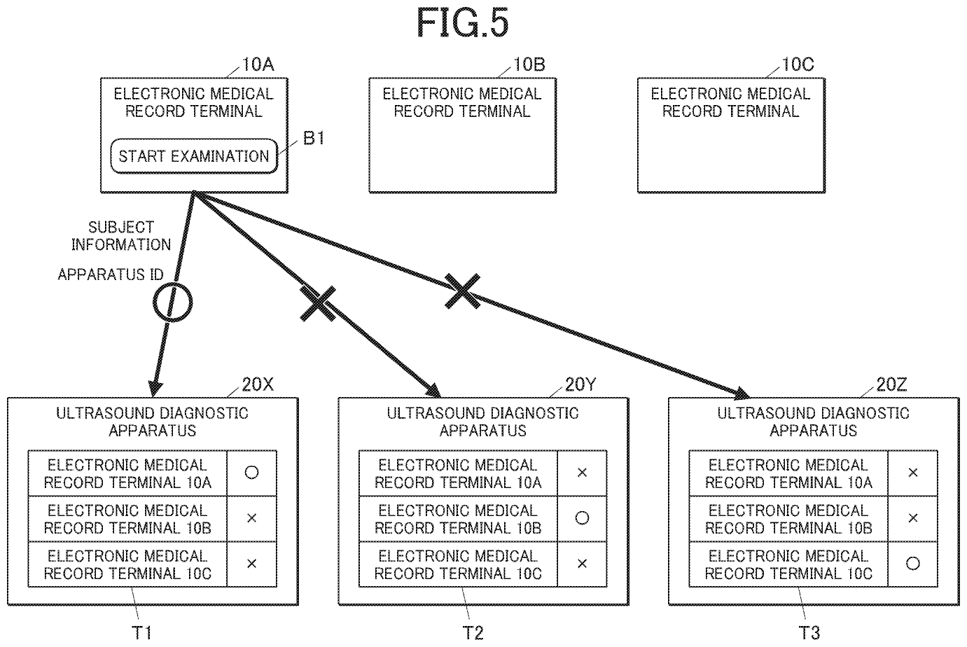

Next, a specific example of the first subject information setting process will be described with reference to FIG. 5.

In the electronic medical record terminal 10A among the electronic medical record terminals 10A, 10B, and 10C, when an “examination start” button “B1” displayed on the display 13 is pressed by an operation through the operation interface 12 while the subject information is displayed on the display 13, the CPU 11 of the electronic medical record terminal 10A transmits the subject information displayed on the display 13 and the apparatus ID of the electronic medical record terminal 10A to the ultrasound diagnostic apparatuses 20X, 20Y, and 20Z through the communicator 14. Destination information such as an IP address for setting the ultrasound diagnostic apparatuses 20X, 20Y, and 20Z as transmission destinations is stored in the storage 16.

A setting table T1 is stored in the storage 28 of the ultrasound diagnostic apparatus 20X. In the setting table T1, it is set that the electronic medical record terminal 10A is an “electronic medical record terminal as an acquisition destination” of the ultrasound diagnostic apparatus 20X (0) and the electronic medical record terminals 10B and 10C are not “electronic medical record terminals as acquisition destinations” of the ultrasound diagnostic apparatus 20X″ (X).

When the subject information and the apparatus ID are received from the electronic medical record terminal 10A, the hardware processor 27 of the ultrasound diagnostic apparatus 20X specifies that the transmission source is the electronic medical record terminal 10A based on the apparatus ID. Referring to the setting table T1, since the electronic medical record terminal 10A is set as an “electronic medical record terminal as an acquisition designation” of the ultrasound diagnostic apparatus 20X, the hardware processor 27 of the ultrasound diagnostic apparatus 20X receives the subject information received from the electronic medical record terminal 10A as an examination target.

A setting table T2 is stored in the storage 28 of the ultrasound diagnostic apparatus 20Y. In the setting table T2, it is set that the electronic medical record terminal 10B is an “electronic medical record terminal as an acquisition destination” of the ultrasound diagnostic apparatus 20Y (O) and the electronic medical record terminals 10A and 10C are not “electronic medical record terminals as acquisition destinations” of the ultrasound diagnostic apparatus 20Y″ (X).

When the subject information and the apparatus ID are received from the electronic medical record terminal 10A, the hardware processor 27 of the ultrasound diagnostic apparatus 20Y specifies that the transmission source is the electronic medical record terminal 10A based on the apparatus ID. Referring to the setting table T2, since the electronic medical record terminal 10A is not set as an “electronic medical record terminal as an acquisition designation” of the ultrasound diagnostic apparatus 20Y, the hardware processor 27 of the ultrasound diagnostic apparatus 20Y discards the subject information received from the electronic medical record terminal 10A.

A setting table T3 is stored in the storage 28 of the ultrasound diagnostic apparatus 20Z. In the setting table T3, it is set that the electronic medical record terminal 10C is an “electronic medical record terminal as an acquisition destination” of the ultrasound diagnostic apparatus 20Z (0) and the electronic medical record terminals 10A and 10B are not “electronic medical record terminals as acquisition destinations” of the ultrasound diagnostic apparatus 20Z″ (X).

When the subject information and the apparatus ID are received from the electronic medical record terminal 10A, the hardware processor 27 of the ultrasound diagnostic apparatus 20Z specifies that the transmission source is the electronic medical record terminal 10A based on the apparatus ID. Referring to the setting table T3, since the electronic medical record terminal 10A is not set as an “electronic medical record terminal as an acquisition designation” of the ultrasound diagnostic apparatus 20Z, the hardware processor 27 of the ultrasound diagnostic apparatus 20Z discards the subject information received from the electronic medical record terminal 10A.

As described above, according to the first embodiment, the electronic medical record terminal 10 transmits the subject information and the apparatus ID of the electronic medical record terminal 10 to all the ultrasound diagnostic apparatuses 20, and on the ultrasound diagnostic apparatus 20 side, the electronic medical record terminal 10 from which the subject information has been acquired is specified based on the apparatus ID. For this reason, it is not necessary to designate one transmission destination and transmit subject information on the electronic medical record terminal 10 side or to receive subject information by limiting to one transmission source on the ultrasound diagnostic apparatus 20 side. Therefore, such a setting can be omitted.

In each ultrasound diagnostic apparatus 20, the “electronic medical record terminal as an acquisition designation” is set in advance. Therefore, it is possible to control whether to use the received subject information or to discard the received subject information by determining whether or not the electronic medical record terminal 10 that has transmitted the subject information and the apparatus ID is an “electronic medical record terminal as an acquisition designation”. As described above, even in a situation in which there are a plurality of ultrasound diagnostic apparatuses 20 and a plurality of electronic medical record terminals 10, the ultrasound diagnostic apparatus 20 can easily acquire subject information from the desired electronic medical record terminal 10 without adding a new hardware configuration. The ultrasound diagnostic apparatus 20 can start an examination using the acquired subject information only when the subject information is acquired from the specific electronic medical record terminal 10.

Specifically, the ultrasound diagnostic apparatus 20 can determine whether or not the electronic medical record terminal 10 that has transmitted the subject information and the apparatus ID is an “electronic medical record terminal as an acquisition designation”, from which the ultrasound diagnostic apparatus 20 should acquire the subject information, based on the setting table stored in the ultrasound diagnostic apparatus 20.

In addition, by changing the setting table, it is possible to easily change the setting of the “electronic medical record terminal as an acquisition designation”.

By using only the subject ID (patient ID) as subject information transmitted from the electronic medical record terminal 10 to the ultrasound diagnostic apparatus 20, security can be improved, for example, leakage of personal information can be prevented.

### Second Embodiment

Next, a second embodiment to which the present invention is applied will be described.

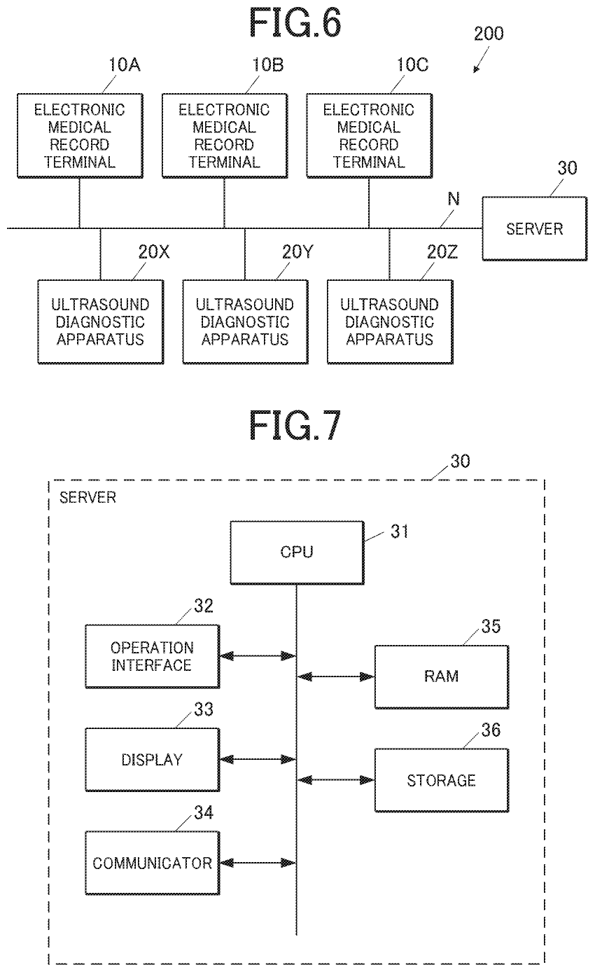

FIG. 6 illustrates a system configuration of a medical examination system 200 according to the second embodiment. As illustrated in FIG. 6, the medical examination system 200 is configured to include electronic medical record terminals 10A, 10B, and 10C as a plurality of information processing apparatuses, ultrasound diagnostic apparatuses 20X, 20Y, and 20Z as a plurality of medical examination apparatuses, and a server 30. The electronic medical record terminals 10A, 10B, and 10C, the ultrasound diagnostic apparatuses 20X, 20Y, and 20Z, and the server 30 are connected to each other through a communication network N, such as a LAN, so that data communication is possible. The medical examination system 200 is installed in a hospital. In the second embodiment, the description of the same configuration as in the first embodiment of the electronic medical record terminal 10 (refer to FIG. 2) and the ultrasound diagnostic apparatus 20 (refer to FIG. 3) will be omitted, and the following description will be given focusing on the characteristic configuration and processing of the second embodiment.

In the first embodiment, a setting table used for determination relevant to an “electronic medical record terminal as an acquisition designation” is stored in each ultrasound diagnostic apparatus 20. In the second embodiment, however, a setting table used for determination relevant to an “electronic medical record terminal as an acquisition designation” is stored in the server 30.

The server 30 is a server that can be accessed from a plurality of ultrasound diagnostic apparatuses 20 in a hospital, and manages the correspondence relationship between the plurality of electronic medical record terminals 10 and the plurality of ultrasound diagnostic apparatuses 20.

FIG. 7 illustrates a functional configuration of the server 30. As illustrated in FIG. 7, the server 30 includes a CPU 31 (second hardware processor), an operation interface 32, a display 33, a communicator 34, a RAM 35, and a storage 36.

The CPU 31 reads various programs such as system programs and processing programs stored in the storage 36 and loads the programs to the RAM 35, and controls the operation of each unit of the server 30 according to the loaded programs.

The operation interface 32 includes a keyboard having cursor keys, character and number input keys, various function keys, and the like, and a pointing device such as a mouse. A press signal of a key pressed on the keyboard and a position input signal by the pointing device are output to the CPU 31 as operation signals.

The display 33 is configured to include a monitor such as an LCD, and displays various screens according to instructions of a display signal input from the CPU 31.

The communicator 34 includes a network interface and the like, and transmits and receives data to and from an external apparatus such as the ultrasound diagnostic apparatus 20 connected through the communication network N.

The RAM 35 is a volatile semiconductor memory, and forms a work area where various programs that can be executed by the CPU 31, input or output data, parameters, and the like, which are read from the storage 36, are temporarily stored in various kinds of processing executed by the CPU 31.

The storage 36 is configured by an HDD, a nonvolatile semiconductor memory, or the like, and stores a system program or various programs executed by the CPU 31.

The storage 36 stores a setting table in which each of the plurality of ultrasound diagnostic apparatuses 20 is associated with each of the plurality of electronic medical record terminals 10. In the second embodiment, the setting table is managed by the server 30, and a common setting table from each ultrasound diagnostic apparatus 20 is referred to.

When a request for transmission of a setting table is received from any one of the plurality of ultrasound diagnostic apparatuses 20, the CPU 31 transmits the setting table to the requesting ultrasound diagnostic apparatus 20.

The communicator 34 (receiver) receives a setting table change instruction from any one of the plurality of ultrasound diagnostic apparatuses 20.

When the setting table change instruction is received through the communicator 34, the CPU 31 changes the correspondence relationship between the plurality of ultrasound diagnostic apparatuses 20 and the plurality of electronic medical record terminals 10 in the setting table.

The communicator 29 of the ultrasound diagnostic apparatus 20 can perform data communication with the server 30.

The hardware processor 27 of the ultrasound diagnostic apparatus 20 sets an “electronic medical record terminal as an acquisition destination” for the host apparatus and other apparatuses, among the plurality of electronic medical record terminals 10, according to an operation through the operation inputter 23. The hardware processor 27 sets an “electronic medical record terminal as an acquisition destination” by changing the setting table stored in the storage 36 of the server 30.

When subject information regarding a subject and an apparatus ID for identifying the electronic medical record terminal 10 are received from any one of the plurality of electronic medical record terminals 10 through the communicator 29, the hardware processor 27 determines whether or not the electronic medical record terminal 10 that has transmitted the received subject information and apparatus ID is an “electronic medical record terminal as an acquisition destination” based on the received apparatus ID.

Based on the information regarding the setting table acquired from the server 30, the hardware processor 27 determines whether or not the electronic medical record terminal 10 as a transmission source is an “electronic medical record terminal as an acquisition destination”. In the second embodiment, the “information regarding the setting table” corresponds to the setting table itself.

Specifically, the hardware processor 27 acquires a setting table from the server 30 through the communicator 29, and determines whether or not the electronic medical record terminal 10 as a transmission source is an “electronic medical record terminal as an acquisition destination” by determining whether or not the ultrasound diagnostic apparatus 20 associated with the electronic medical record terminal 10 as a transmission source in the setting table is the host apparatus.

When it is determined that the electronic medical record terminal 10 as a transmission source is an “electronic medical record terminal as an acquisition destination”, the hardware processor 27 sets the received subject information as subject information regarding a subject to be examined by the ultrasound diagnostic apparatus 20 (examination data generator).

Next, the operation of the second embodiment will be described.

The method of transmitting subject information displayed on the display 13 and the apparatus ID for identifying the electronic medical record terminal 10 from any one of the plurality of electronic medical record terminals 10 to all the ultrasound diagnostic apparatuses 20 in the medical examination system 200 is the same as that in the first embodiment.

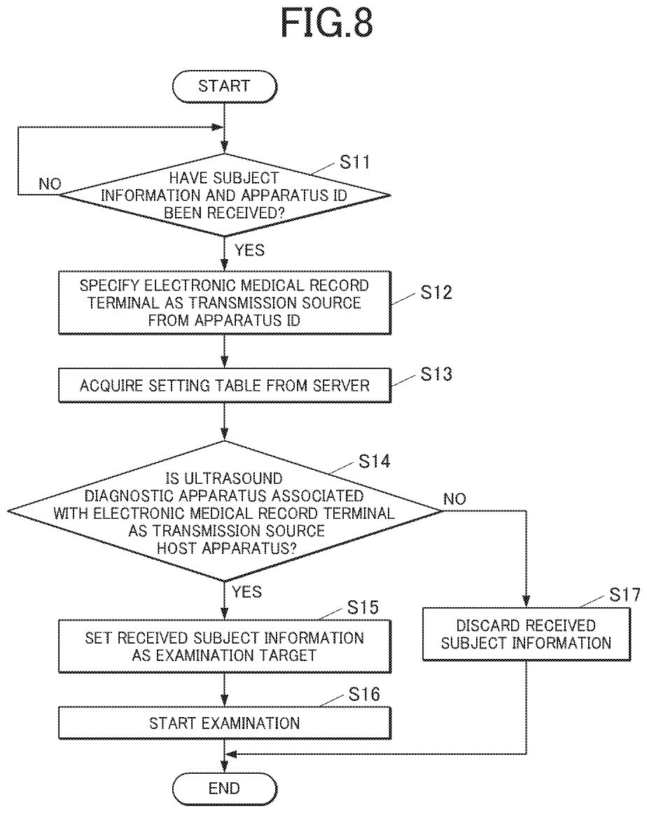

FIG. 8 is a flowchart illustrating a second subject information setting process executed by each ultrasound diagnostic apparatus 20. This process is realized by software processing in cooperation with the CPU of the hardware processor 27 and the program stored in the ROM.

The processing of steps S11 to S12 is the same as the processing of steps S1 to S2 in the first subject information setting process (refer to FIG. 4), and accordingly, the description thereof will be omitted.

Then, the hardware processor 27 acquires a setting table from the server 30 through the communicator 29 (step S13). Specifically, the hardware processor 27 transmits a setting table transmission request to the server 30 through the communicator 29. In the server 30, when the setting table transmission request is received from the ultrasound diagnostic apparatus 20, the CPU 31 reads the setting table from the storage 36 and transmits the read setting table to the requesting ultrasound diagnostic apparatus 20. The requesting ultrasound diagnostic apparatus 20 receives the setting table from the server 30.

Then, the hardware processor 27 determines whether or not the ultrasound diagnostic apparatus 20 associated with the electronic medical record terminal 10 that has transmitted the subject information and the apparatus ID is the host apparatus with reference to the setting table acquired from the server 30 (step S14).

When the ultrasound diagnostic apparatus 20 associated with the electronic medical record terminal 10 as a transmission source is the host apparatus (step S14; YES), the hardware processor 27 sets the received subject information as subject information regarding a subject to be examined (step S15), and starts an examination (step S16).

When the ultrasound diagnostic apparatus 20 associated with the electronic medical record terminal 10 as a transmission source is not the host apparatus in step S14 (step S14; NO), the hardware processor 27 discards the received subject information (step S17).

After step S16 or step S17, the second subject information setting process ends.

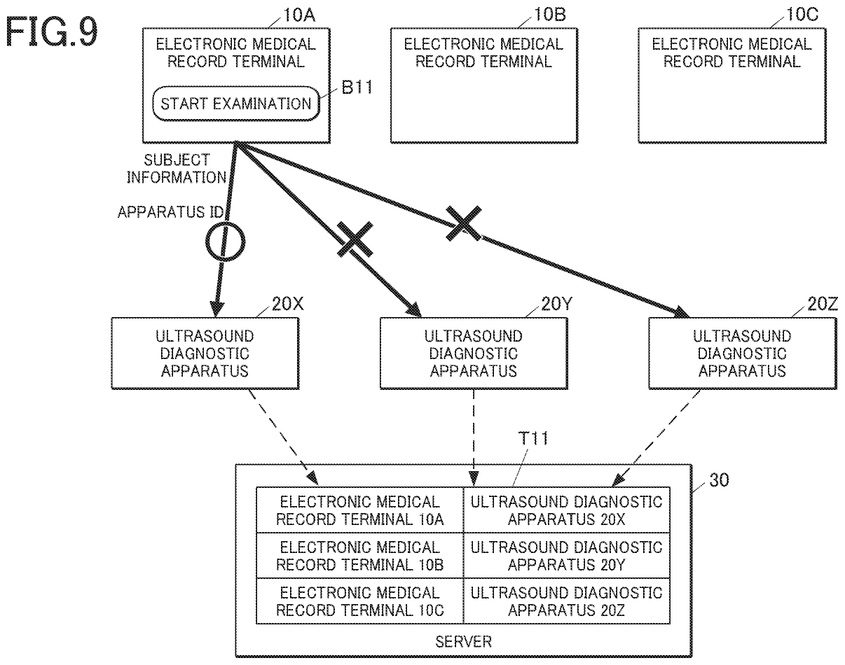

Next, a specific example of the second subject information setting process will be described with reference to FIG. 9.

In the electronic medical record terminal 10A among the electronic medical record terminals 10A, 10B, and 10C, when an “examination start” button B11 displayed on the display 13 is pressed by an operation through the operation interface 12, the CPU 11 of the electronic medical record terminal 10A transmits the subject information displayed on the display 13 and the apparatus ID of the electronic medical record terminal 10A to the ultrasound diagnostic apparatuses 20X, 20Y, and 20Z through the communicator 14.

A setting table T11 is stored in the storage 36 of the server 30. In the setting table T11, it is set that the electronic medical record terminal 10A is an “electronic medical record terminal as an acquisition designation” of the ultrasound diagnostic apparatus 20X, the electronic medical record terminal 10B is an “electronic medical record terminal as an acquisition designation” of the ultrasound diagnostic apparatus 20Y, and the electronic medical record terminal 10C is an “electronic medical record terminal as an acquisition designation” of the ultrasound diagnostic apparatus 20Z.

When the subject information and the apparatus ID are received from the electronic medical record terminal 10A, the hardware processor 27 of the ultrasound diagnostic apparatus 20X specifies that the transmission source is the electronic medical record terminal 10A based on the apparatus ID. Referring to the setting table T11 acquired from the server 30, since the ultrasound diagnostic apparatus 20 associated with the electronic medical record terminal 10A as a transmission source is the ultrasound diagnostic apparatus 20X, the hardware processor 27 of the ultrasound diagnostic apparatus 20X sets the subject information received from the electronic medical record terminal 10A as an examination target.

When the subject information and the apparatus ID are received from the electronic medical record terminal 10A, the hardware processor 27 of the ultrasound diagnostic apparatus 20Y specifies that the transmission source is the electronic medical record terminal 10A based on the apparatus ID. Referring to the setting table T11 acquired from the server 30, since the ultrasound diagnostic apparatus 20 associated with the electronic medical record terminal 10A as a transmission source is not the ultrasound diagnostic apparatus 20Y, the hardware processor 27 of the ultrasound diagnostic apparatus 20Y discards the subject information received from the electronic medical record terminal 10A.

When the subject information and the apparatus ID are received from the electronic medical record terminal 10A, the hardware processor 27 of the ultrasound diagnostic apparatus 20Z specifies that the transmission source is the electronic medical record terminal 10A based on the apparatus ID. Referring to the setting table T11 acquired from the server 30, since the ultrasound diagnostic apparatus 20 associated with the electronic medical record terminal 10A as a transmission source is not the ultrasound diagnostic apparatus 20Z, the hardware processor 27 of the ultrasound diagnostic apparatus 20Z discards the subject information received from the electronic medical record terminal 10A.

As described above, according to the second embodiment, even in a situation in which there are a plurality of ultrasound diagnostic apparatuses 20 and a plurality of electronic medical record terminals 10, the ultrasound diagnostic apparatus 20 can easily acquire subject information from the desired electronic medical record terminal 10 without adding a new hardware configuration. The ultrasound diagnostic apparatus 20 can start an examination using the acquired subject information only when the subject information is acquired from the specific electronic medical record terminal 10.

Specifically, the ultrasound diagnostic apparatus 20 can acquire a setting table from the server 30 and determine whether or not the electronic medical record terminal 10 as a transmission source is an “electronic medical record terminal as an acquisition destination” by determining whether or not the ultrasound diagnostic apparatus 20 associated with the electronic medical record terminal 10 as a transmission source in the setting table is the host apparatus. By sharing the setting table among the ultrasound diagnostic apparatuses 20, for example, it is possible to change the settings for all the ultrasound diagnostic apparatuses 20 from one ultrasound diagnostic apparatus 20.

In addition, by changing the setting table, it is possible to easily change the setting of the “electronic medical record terminal as an acquisition designation”.

In the second embodiment, the setting table is acquired from the server 30 at the timing when the ultrasound diagnostic apparatus 20 receives the subject information and the apparatus ID. However, at a timing when there is a change in the setting table managed by the server 30, the setting table may be transmitted from the server 30 to each ultrasound diagnostic apparatus 20, and the latest setting table may always be stored in each ultrasound diagnostic apparatus 20.

Alternatively, the electronic medical record terminal 10 may transmit subject information and the apparatus ID only to the specific ultrasound diagnostic apparatus 20. Then, when the specific ultrasound diagnostic apparatus 20 determines that the electronic medical record terminal 10 that has transmitted the subject information does not match the “electronic medical record terminal as an acquisition designation”, the subject information may be discarded after the subject information is transmitted to other ultrasound diagnostic apparatuses 20 associated with the electronic medical record terminal 10 as a transmission source. In this case, the subject information may be directly transmitted to the other ultrasound diagnostic apparatuses 20, or the electronic medical record terminal 10 as a transmission source may be requested to retransmit the subject information to the other ultrasound diagnostic apparatuses 20.

The server 30 can also be accessed from the electronic medical record terminal 10, so that the setting table in the server 30 can be changed according to an operation through the electronic medical record terminal 10.

The electronic medical record terminal 10 may specify the ultrasound diagnostic apparatus 20 associated with the electronic medical record terminal 10 with reference to the setting table in the server 30, and the ultrasound diagnostic apparatus 20 as a transmission destination may be set so as to transmit the subject information only to the specified ultrasound diagnostic apparatus 20. By referring to the same setting table on the electronic medical record terminal 10 side and the ultrasound diagnostic apparatus 20 side, useless data transmission can be eliminated. As a result, the load can be reduced.

any one of the plurality of ultrasound diagnostic apparatuses 20 or any one of the plurality of electronic medical record terminals 10 may have the function of the server 30 that manages a setting table. In this case, it is assumed that a setting table whose setting is changed can always be used by the ultrasound diagnostic apparatus 20 that does not have the function of the server 30.

### MODIFICATION EXAMPLES

Next, a modification example of the second embodiment will be described. In the second embodiment, a case where the ultrasound diagnostic apparatus 20 acquires a setting table from the server 30 has been described. However, in the modification example, the ultrasound diagnostic apparatus 20 acquires a determination result based on the setting table from the server 30. Only the differences from the second embodiment will be described below.

When the apparatus ID of the electronic medical record terminal 10 that has transmitted subject information and the apparatus ID to any one of the plurality of ultrasound diagnostic apparatuses 20 is received from the ultrasound diagnostic apparatus 20, the CPU 31 of the server 30 determines whether or not the ultrasound diagnostic apparatus 20 associated with the electronic medical record terminal 10 as a transmission source is the requesting ultrasound diagnostic apparatus 20, which has transmitted the apparatus ID to the server 30, based on the received apparatus ID with reference to the setting table stored in the storage 36, and transmits the determination result to the requesting ultrasound diagnostic apparatus 20.

Based on the information regarding the setting table acquired from the server 30, the hardware processor 27 of the ultrasound diagnostic apparatus 20 determines whether or not the electronic medical record terminal 10 as a transmission source is an “electronic medical record terminal as an acquisition destination”. In the modification example, the “information regarding the setting table” corresponds to a determination result based on the setting table.

Specifically, the hardware processor 27 transmits the apparatus ID of the electronic medical record terminal 10 as a transmission source to the server 30 through the communicator 29, acquires a determination result as to whether or not the ultrasound diagnostic apparatus 20 associated with the electronic medical record terminal 10 as a transmission source in the setting table is the host apparatus (ultrasound diagnostic apparatus 20 that has transmitted the apparatus ID to the server 30) from the server 30, and determines whether or not the electronic medical record terminal 10 as a transmission source is an “electronic medical record terminal as an acquisition designation” based on the determination result.

In the modification example, in the second subject information setting process illustrated in FIG. 8, processing of steps S13 and S14 is different from that in the second embodiment.

In the modification example, instead of step S13, the hardware processor 27 of the ultrasound diagnostic apparatus 20 acquires a determination result based on the setting table from the server 30 through the communicator 29. Specifically, the hardware processor 27 transmits the apparatus ID of the electronic medical record terminal 10, which has transmitted the subject information and the apparatus ID, to the server 30 through the communicator 29.

In the server 30, when the apparatus ID is received from the ultrasound diagnostic apparatus 20, the CPU 31 reads a setting table from the storage 36, determines whether or not the ultrasound diagnostic apparatus 20 associated with the electronic medical record terminal 10 as a transmission source is the requesting ultrasound diagnostic apparatus 20, which has transmitted the apparatus ID to the server 30, based on the received apparatus ID with reference to the read setting table, and transmits the determination result to the requesting ultrasound diagnostic apparatus 20.

Then, the requesting ultrasound diagnostic apparatus 20 receives a determination result based on the setting table from the server 30.

Then, instead of step S14, the hardware processor 27 of the ultrasound diagnostic apparatus 20 determines whether or not the electronic medical record terminal 10 that has transmitted the subject information and the apparatus ID, is an “electronic medical record terminal as an acquisition destination” based on the determination result acquired from the server 30. Specifically, when the ultrasound diagnostic apparatus 20 associated with the electronic medical record terminal 10 as a transmission source is the requesting ultrasound diagnostic apparatus 20, the electronic medical record terminal 10 as a transmission source is set as an “electronic medical record terminal as an acquisition destination”. On the other hand, when the ultrasound diagnostic apparatus 20 associated with the electronic medical record terminal 10 as a transmission source is not the requesting ultrasound diagnostic apparatus 20, the electronic medical record terminal 10 as a transmission source is not set as an “electronic medical record terminal as an acquisition destination”.

When the electronic medical record terminal 10 as a transmission source is an “electronic medical record terminal as an acquisition destination”, the hardware processor 27 of the ultrasound diagnostic apparatus 20 performs processing of steps S15 and S16.

When the electronic medical record terminal 10 as a transmission source is not an “electronic medical record terminal as an acquisition destination”, the hardware processor 27 of the ultrasound diagnostic apparatus 20 performs processing of step S17.

According to the modification example, when the apparatus ID of the electronic medical record terminal 10 as a transmission source is received from the ultrasound diagnostic apparatus 20, the server 30 can determine whether or not the ultrasound diagnostic apparatus 20 associated with the electronic medical record terminal 10 as a transmission source is the requesting ultrasound diagnostic apparatus 20, which has transmitted the apparatus ID to the server 30, based on the received apparatus ID with reference to the setting table and transmit the determination result to the requesting ultrasound diagnostic apparatus 20.

The ultrasound diagnostic apparatus 20 can acquire a determination result based on the setting table from the server 30 and determine whether or not the electronic medical record terminal 10 as a transmission source is an “electronic medical record terminal as an acquisition destination” based on the determination result.

### Third Embodiment

Next, a third embodiment to which the present invention is applied will be described. Since a medical examination system according to the third embodiment has the same configuration as the medical examination system 200 illustrated in the second embodiment, FIGS. 2, 3, 6, and 7 are used, and illustration and description thereof will be omitted. Hereinafter, the characteristic configuration and processing of the third embodiment will be described.

The hardware processor 27 of the ultrasound diagnostic apparatus 20 makes a transition to an acquisition destination change mode in response to an operation through the operation inputter 23.

When subject information and an apparatus ID for identifying the electronic medical record terminal 10 are received from any one of the electronic medical record terminals 10 through the communicator 29 in the acquisition destination change mode, the hardware processor 27 sets the electronic medical record terminal 10 as an “electronic medical record terminal as an acquisition designation”.

When the subject information is received from any one of the electronic medical record terminals 10 through the communicator 29 in the acquisition destination change mode, the hardware processor 27 ends the acquisition destination change mode.

Next, the operation of the third embodiment will be described.

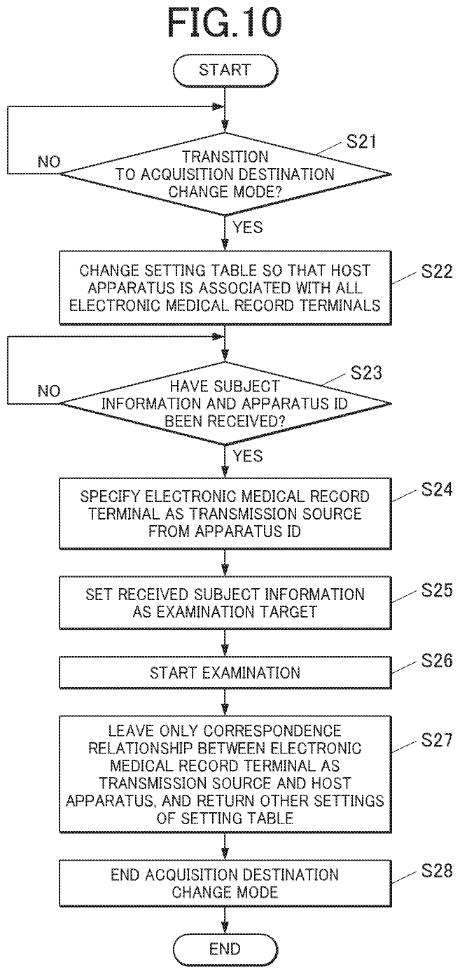

FIG. 10 is a flowchart illustrating an acquisition destination change mode process executed by the ultrasound diagnostic apparatus 20 that changes an acquisition destination. This process is realized by software processing in cooperation with the CPU of the hardware processor 27 and the program stored in the ROM.

First, the hardware processor 27 determines whether or not there is an instruction to transition to the acquisition destination change mode by an operation through the operation inputter 23 (step S21). When there is no instruction to transition to the acquisition destination change mode (step S21; NO), the process returns to step S21.

When there is an instruction to transition to the acquisition destination change mode in step S21 (step S21; YES), the hardware processor 27 accesses the server 30 through the communicator 29 and changes the setting table of the server 30 so that the host apparatus is associated with all the electronic medical record terminals 10 (step S22). Specifically, the hardware processor 27 transmits a setting table change instruction to the server 30, and the CPU 31 of the server 30 changes the setting table in the server 30 based on the change instruction. Alternatively, the hardware processor 27 may acquire a setting table from the server 30, change the setting table in the ultrasound diagnostic apparatus 20, and transmit the changed setting table to the server 30.

The subject information displayed on the display 13 and the apparatus ID for identifying the electronic medical record terminal 10 are transmitted from any one of the plurality of electronic medical record terminals 10 to all the ultrasound diagnostic apparatuses 20 in the medical examination system.

The processing of steps S23 and S24 is the same as the processing of steps S1 and S2 in the first subject information setting process (refer to FIG. 4), and accordingly, the description thereof will be omitted.

Then, the hardware processor 27 sets the received subject information as subject information regarding a subject to be examined (step S25), and starts an examination (step S26).

Then, the hardware processor 27 accesses the server 30 through the communicator 29, and changes the setting table of the server 30 so that only the correspondence relationship between the electronic medical record terminal 10, which has transmitted the subject information and the apparatus ID, and the host apparatus is left and the other settings are returned to the state before the processing of step S22 (step S27). Specifically, the hardware processor 27 transmits a setting table change instruction to the server 30, and the CPU 31 of the server 30 changes the setting table in the server 30 based on the change instruction. Alternatively, the hardware processor 27 may acquire a setting table from the server 30, change the setting table in the ultrasound diagnostic apparatus 20, and transmit the changed setting table to the server 30.

Next, the hardware processor 27 ends the acquisition destination change mode (step S28).

Thus, the acquisition destination change mode process ends.

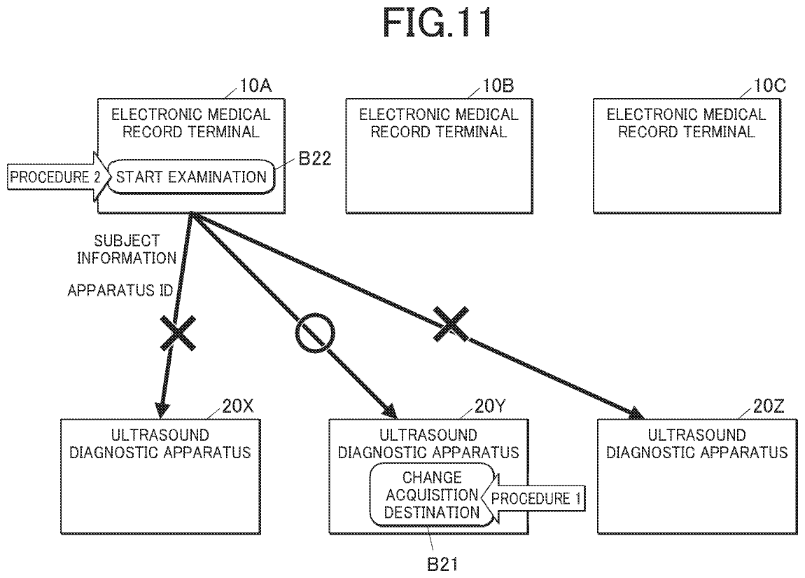

Next, a specific example of the acquisition destination change mode process will be described with reference to FIG. 11.

First, in the ultrasound diagnostic apparatus 20Y among the ultrasound diagnostic apparatuses 20X, 20Y, and 20Z, when an “acquisition destination change” button B21 displayed on the display 26 is pressed by an operation through the operation inputter 23 (procedure 1), the ultrasound diagnostic apparatus 20Y transitions to the acquisition destination change mode.

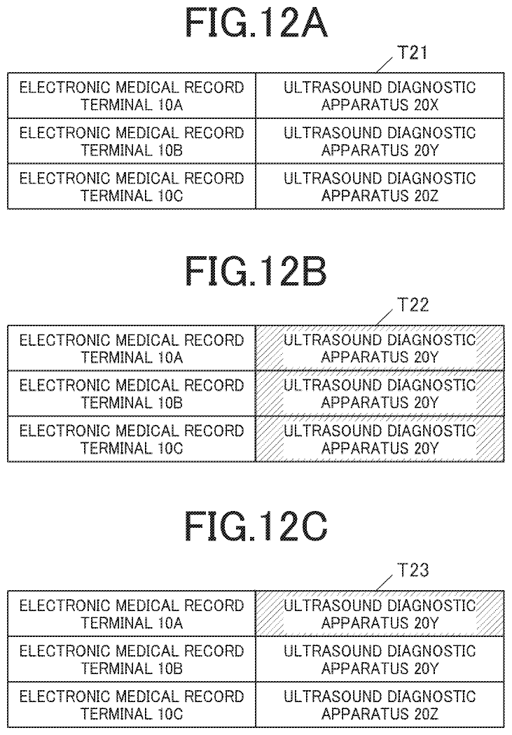

FIG. 12A illustrates an example of a setting table T21 before the transition to the acquisition destination change mode. In FIG. 12A, the ultrasound diagnostic apparatuses 20X, 20Y, and 20Z are associated with the electronic medical record terminals 10A, 10B, and 10C, respectively.

When the “acquisition destination change” button B21 is pressed in this state, the hardware processor 27 of the ultrasound diagnostic apparatus 20Y changes the setting table to the state of a setting table T22 illustrated in FIG. 12B. In FIG. 12B, the ultrasound diagnostic apparatus 20Y is associated with all the electronic medical record terminals 10A, 10B, and 10C. That is, the hardware processor 27 temporarily changes the setting table so that the subject information transmitted from the electronic medical record terminals 10A, 10B, and 10C is set only in the ultrasound diagnostic apparatus 20Y and the ultrasound diagnostic apparatus 20Y can acquire the subject information from all the electronic medical record terminals 10A, 10B, and 10C.

Then, as illustrated in FIG. 11, in the electronic medical record terminal 10A among the electronic medical record terminals 10A, 10B, and 10C, when an “examination start” button B22 displayed on the display 13 is pressed by an operation through the operation interface 12 (procedure 2), the CPU 11 of the electronic medical record terminal 10A transmits the subject information displayed on the display 13 and the apparatus ID of the electronic medical record terminal 10A to the ultrasound diagnostic apparatuses 20X, 20Y, and 20Z through the communicator 14.

When the subject information and the apparatus ID are received from the electronic medical record terminal 10A, the hardware processor 27 of the ultrasound diagnostic apparatus 20Y specifies that the transmission source is the electronic medical record terminal 10A based on the apparatus ID. Referring to the setting table T22 in the server 30, since the electronic medical record terminal 10A as a transmission source is associated with the ultrasound diagnostic apparatus 20Y, the hardware processor 27 of the ultrasound diagnostic apparatus 20Y sets the subject information received from the electronic medical record terminal 10A as an examination target.

On the other hand, when the subject information and the apparatus ID are received from the electronic medical record terminal 10A, the hardware processor 27 of the ultrasound diagnostic apparatus 20X specifies that the transmission source is the electronic medical record terminal 10A based on the apparatus ID. Referring to the setting table T22 in the server 30, since the ultrasound diagnostic apparatus 20 associated with the electronic medical record terminal 10A as a transmission source is not the ultrasound diagnostic apparatus 20X, the hardware processor 27 of the ultrasound diagnostic apparatus 20X discards the subject information received from the electronic medical record terminal 10A.

The processing in the ultrasound diagnostic apparatus 20Z is the same as that of the ultrasound diagnostic apparatus 20X.

Thereafter, the hardware processor 27 of the ultrasound diagnostic apparatus 20Y changes the setting table so that only the correspondence relationship between the electronic medical record terminal 10A as a transmission source and the host apparatus (ultrasound diagnostic apparatus 20Y) is left and the other settings are returned to the state of the setting table T21 in FIG. 12A, as in a setting table T23 illustrated in FIG. 12C.

As described above, according to the third embodiment, when the subject information and the apparatus ID are received from any one of the electronic medical record terminals 10 in the acquisition destination change mode, the ultrasound diagnostic apparatus 20 sets the electronic medical record terminal 10 as an “electronic medical record terminal as an acquisition destination” for the ultrasound diagnostic apparatus 20. Therefore, it is possible to easily change the acquisition destination of the subject information.

In the third embodiment, transition to the acquisition destination change mode is made by pressing the “acquisition destination change” button B21. However, transition to the acquisition destination change mode may be triggered by execution of predetermined processing other than user input or by detection of a change in status.

### Fourth Embodiment

Next, a fourth embodiment to which the present invention is applied will be described.

Since a medical examination system according to the fourth embodiment has the same configuration as the medical examination system according to the third embodiment, illustration and description thereof will be omitted. Hereinafter, the characteristic configuration and processing of the fourth embodiment will be described.

The fourth embodiment is based on the acquisition destination change mode process in the third embodiment.

The hardware processor 27 of the ultrasound diagnostic apparatus 20 determines whether or not two or more electronic medical record terminals 10 associated with the same ultrasound diagnostic apparatus 20 are present in the setting table stored in the storage 36 of the server 30.

When it is determined that there are two or more electronic medical record terminals 10 associated with the same ultrasound diagnostic apparatus 20, the hardware processor 27 changes the setting table so that one of the two or more electronic medical record terminals 10 is associated with the ultrasound diagnostic apparatus 20 associated with the two or more electronic medical record terminals 10.

Specifically, the hardware processor 27 changes the setting table so that the electronic medical record terminal 10 whose correspondence relationship with the same ultrasound diagnostic apparatus 20 is set last, among the two or more electronic medical record terminals 10, is associated with the ultrasound diagnostic apparatus 20 associated with the two or more electronic medical record terminals 10.

When it is determined that there are two or more electronic medical record terminals 10 associated with the same ultrasound diagnostic apparatus 20, the hardware processor 27 notifies the user of the determination result.

The hardware processor 27 causes the user to select whether or not to give priority to the setting of the electronic medical record terminal 10 whose correspondence relationship with the same ultrasound diagnostic apparatus 20 is set last among the two or more electronic medical record terminals 10. Specifically, the hardware processor 27 causes the display 26 to display a screen for selecting whether or not to give priority to the setting of the electronic medical record terminal 10 set last, and receives the user's selection by an operation through the operation inputter 23 (selector).

When giving priority to the setting of the electronic medical record terminal 10 whose correspondence relationship with the same ultrasound diagnostic apparatus 20 is set last is selected by the operation inputter 23, the hardware processor 27 changes the setting table so that the electronic medical record terminal 10 whose correspondence relationship with the same ultrasound diagnostic apparatus 20 is set last is associated with the ultrasound diagnostic apparatus 20 associated with two or more electronic medical record terminals 10.

Next, the operation of the fourth embodiment will be described.

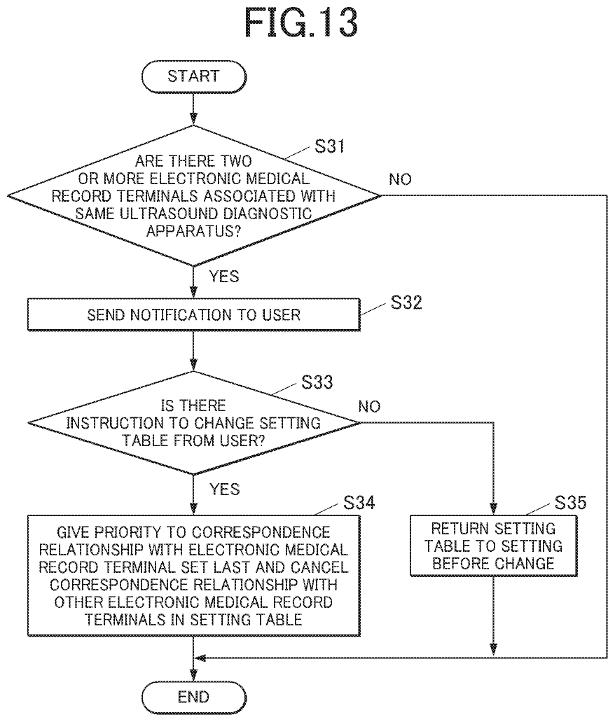

FIG. 13 is a flowchart illustrating a setting table correction process executed by the ultrasound diagnostic apparatus 20 that has executed the acquisition destination change mode process (refer to FIG. 10). This process is a process performed subsequent to the acquisition destination change mode process, and is realized by software processing in cooperation with the CPU of the hardware processor 27 and the program stored in the ROM.

As a result of the acquisition destination change mode process, the hardware processor 27 determines whether or not two or more electronic medical record terminals 10 associated with the same ultrasound diagnostic apparatus 20 are present in the setting table in the server 30 (step S31). Specifically, the hardware processor 27 accesses the server 30 through the communicator 29, acquires a setting table from the server 30, and performs the above-described determination for the acquired setting table.

When there are two or more electronic medical record terminals 10 associated with the same ultrasound diagnostic apparatus 20 (step S31; YES), the hardware processor 27 displays the determination result on the display 26, and notifies the user that there are two or more electronic medical record terminals 10 associated with the same ultrasound diagnostic apparatus 20 (step S32).

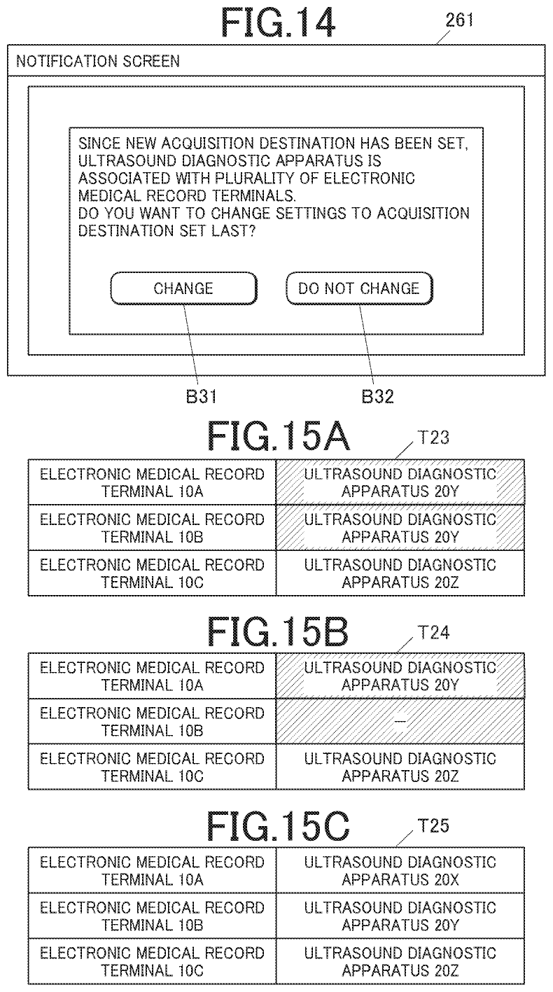

FIG. 14 illustrates an example of a notification screen 261 displayed on the display 26 of the ultrasound diagnostic apparatus 20. The notification screen 261 includes a message, which indicates that the ultrasound diagnostic apparatus 20 being operated is associated with a plurality of electronic medical record terminals 10 since a new acquisition destination has been set in the ultrasound diagnostic apparatus 20 being operated, and a question as to whether or not to change the setting in the setting table so that the ultrasound diagnostic apparatus 20 is associated with only the electronic medical record terminal 10 as an acquisition destination set last. In addition, the notification screen 261 includes a “change” button B31 and a “do not change” button B32. The “change” button B31 is a button for giving an instruction to leave only the correspondence relationship between the ultrasound diagnostic apparatus 20 being operated and the electronic medical record terminal 10 set last and cancel the correspondence relationship between the ultrasound diagnostic apparatus 20 being operated and the other electronic medical record terminals 10. The “do not change” button B32 is a button for giving an instruction to return the setting table to the state before changing the acquisition destination in the ultrasound diagnostic apparatus 20 being operated.

Then, the hardware processor 27 determines whether or not there is an instruction to change the setting table by the user's operation through the operation inputter 23 (step S33).

When there is an instruction to change the setting table (step S33; YES), that is, when the “change” button B31 on the notification screen 261 is pressed, the hardware processor 27 gives priority to the correspondence relationship with the electronic medical record terminal 10 set last, among the correspondence relationships for two or more electronic medical record terminals 10 associated with the same ultrasound diagnostic apparatus 20, and cancels the correspondence relationship with the other electronic medical record terminals 10 in the setting table (step S34). Specifically, the hardware processor 27 leaves only the correspondence relationship with the electronic medical record terminal 10 set last, among the correspondence relationships between the host apparatus and the plurality of electronic medical record terminals 10, and leaves the ultrasound diagnostic apparatuses 20 corresponding to the other electronic medical record terminals 10 blank. The hardware processor 27 transmits a setting table change instruction to the server 30, and the CPU 31 of the server 30 changes the setting table in the server 30 based on the change instruction. Alternatively, the hardware processor 27 may acquire a setting table from the server 30, change the setting table in the ultrasound diagnostic apparatus 20, and transmit the changed setting table to the server 30.

When there is no instruction to change the setting table in step S33 (step S33; NO), that is, when the “do not change” button B32 on the notification screen 261 is pressed, the hardware processor 27 returns the setting table to the setting before the change (step S35). That is, the hardware processor 27 returns to the state of the setting table before the acquisition destination change mode process. The hardware processor 27 transmits an instruction to return the setting table to the setting before the change to the server 30, and the CPU 31 of the server 30 changes the setting table in the server 30 based on the instruction. Alternatively, the hardware processor 27 may acquire the setting table from the server 30, return the setting table to the setting before the change in the ultrasound diagnostic apparatus 20, and transmit the setting table after the return to the server 30.

After step S34, after step S35, or in step S31, when there are no two or more electronic medical record terminals 10 associated with the same ultrasound diagnostic apparatus 20 (step S31; NO), the setting table correction process ends.

FIG. 15A illustrates the setting table T23 that has been changed in the order illustrated in FIGS. 12A to 12C as a result of the acquisition destination change mode process (FIG. 10). In FIG. 15A, the same ultrasound diagnostic apparatus 20Y is associated with two electronic medical record terminals 10 of the electronic medical record terminal 10A and the electronic medical record terminal 10B. When the “change” button B31 is pressed in this state, the hardware processor 27 of the ultrasound diagnostic apparatus 20Y leaves the correspondence relationship between the ultrasound diagnostic apparatus 20Y and the electronic medical record terminal 10A set last for the ultrasound diagnostic apparatus 20Y and cancels the ultrasound diagnostic apparatus 20Y associated with the electronic medical record terminal 10B, as in a setting table T24 illustrated in FIG. 15B. On the other hand, when the “do not change” button B32 is pressed in the state of FIG. 15A, the hardware processor 27 of the ultrasound diagnostic apparatus 20Y returns the setting table to the first state illustrated in FIG. 12A as in a setting table T25 illustrated in FIG. 15C.

As described above, according to the fourth embodiment, when a situation in which the same ultrasound diagnostic apparatus 20 is associated with a plurality of electronic medical record terminals 10 occurs in the setting table, the situation can be resolved. That is, the setting table can be changed so that the setting in the table setting is always exclusive.

Specifically, the setting table can be changed so that the electronic medical record terminal 10 whose correspondence relationship with the same ultrasound diagnostic apparatus 20 is set last is left.

Before changing the setting table, when the user is notified that the same ultrasound diagnostic apparatus 20 is associated with a plurality of electronic medical record terminals 10, the user is made to select whether or not to give priority to the setting of the electronic medical record terminal 10 set last, and the user desires to give priority to the setting of the electronic medical record terminal 10 set last, the setting table can be automatically changed.

In the setting table correction process (see FIG. 13), when there are two or more electronic medical record terminals 10 associated with the same ultrasound diagnostic apparatus 20 (step S31; YES), the user is notified in step S32. Without notification, the process may proceed to step S34 without waiting for an operation through the user in step S33, and the setting table may be automatically corrected.

In the fourth embodiment, the case has been described in which the setting table correction process (refer to FIG. 13) is executed in the ultrasound diagnostic apparatus 20 that gives an instruction to change the acquisition destination, but the setting table correction process may be executed on the server 30 side. However, notification to the user in step S32 or reception of an instruction in step S33 is performed in the ultrasound diagnostic apparatus 20.

### Fifth Embodiment

Next, a fifth embodiment to which the present invention is applied will be described.

Since a medical examination system according to the fifth embodiment has the same configuration as the medical examination system 100 illustrated in the first embodiment, FIGS. 1 to 3 are used, and illustration and description thereof will be omitted. Hereinafter, the characteristic configuration and processing of the fifth embodiment will be described.

In each ultrasound diagnostic apparatus 20, behavior when another subject information is received while certain subject information is set and examined is set in advance.

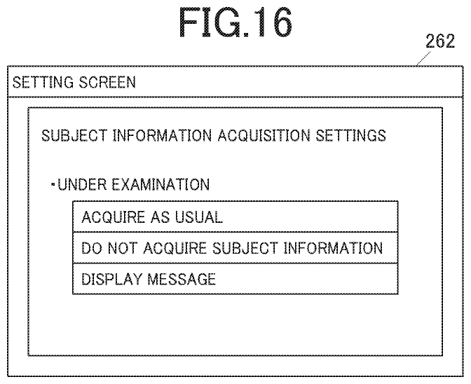

FIG. 16 illustrates an example of a setting screen 262 displayed on the display 26 of the ultrasound diagnostic apparatus 20.

On the setting screen 262, options such as “acquire as usual”, “do not acquire subject information”, and “display message” are displayed as subject information acquisition settings in the examination, so that the user can set one of these. The hardware processor 27 stores the content set as the subject information acquisition settings in the examination in the storage 28.

Next, the operation of the fifth embodiment will be described.

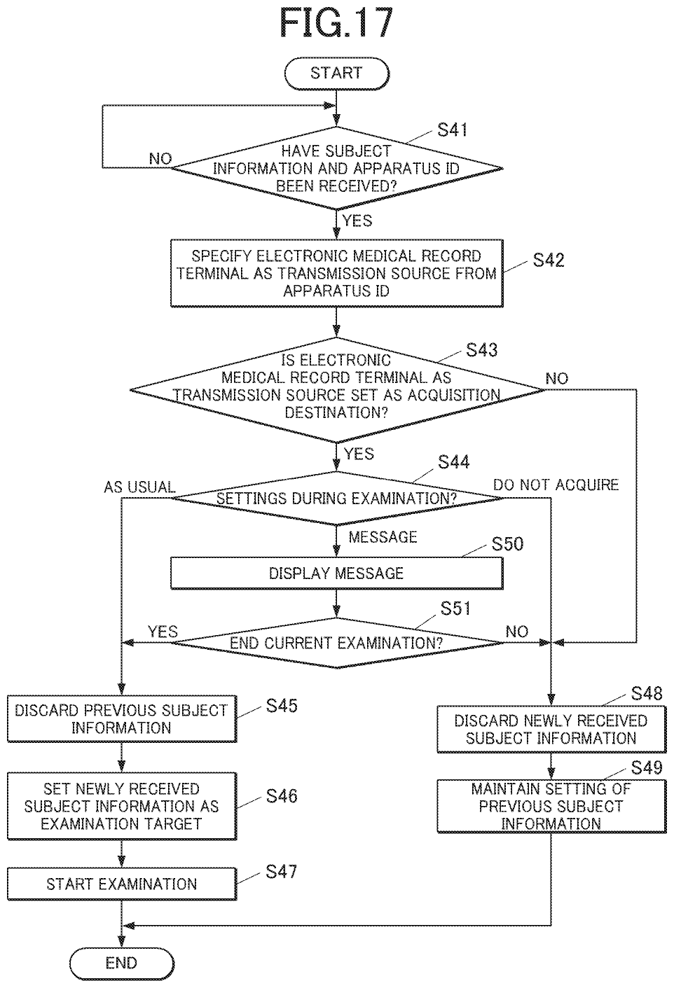

FIG. 17 is a flowchart illustrating a third subject information setting process executed by each ultrasound diagnostic apparatus 20. The third subject information setting process is a process executed after step S5 of the first subject information setting process (refer to FIG. 4), and is realized by software processing in cooperation with the CPU of the hardware processor 27 and the program stored in and ROM.

The processing from step S41 to step S43 is the same as the processing from step S1 to step S3 of the first subject information setting processing (see FIG. 4), and thus description thereof is omitted.

When the electronic medical record terminal 10 as a transmission source is set as an “electronic medical record terminal as an acquisition destination” in step S43 (step S43; YES), the hardware processor 27 determines which of “acquire as usual”, “do not acquire subject information”, and “display message” the subject information acquisition setting in the examination stored in the storage 28 is (step S44).

When the subject information acquisition setting in the examination is “acquire as usual” (step S44; as usual), the hardware processor 27 discards “previous subject information” set in step S4 of the first subject information setting process (step S45).

Then, the hardware processor 27 sets “subject information newly received” in step S41 as subject information regarding a subject to be examined (step S46), and starts an examination (step S47).

When the subject information acquisition setting in the examination is “do not acquire subject information” in step S44 (step S44; do not acquire), the hardware processor 27 discards “subject information newly received” in step S41 (step S48), and maintains the setting of “previous subject information” set in step S4 of the first subject information setting process (step S49).

When the subject information acquisition setting in the examination is “display message” in step S44 (step S44; message), the hardware processor 27 causes the display 26 to display a message indicating that new subject information has been acquired during the examination (step S50).

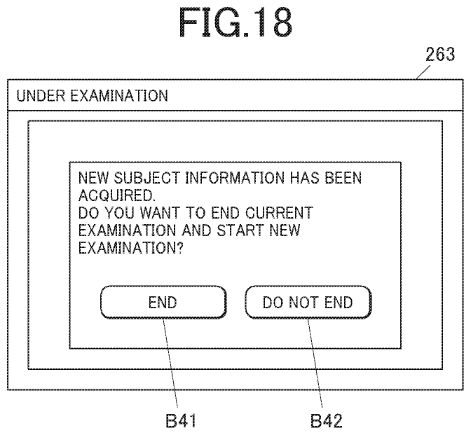

FIG. 18 illustrates an example of a warning screen 263 displayed on the display 26 of the ultrasound diagnostic apparatus 20. The warning screen 263 includes a message indicating that new subject information has been acquired and a question as to whether or not to end the current examination and start a new examination in the ultrasound diagnostic apparatus 20 being operated. The warning screen 263 includes an “end” button B41 and a “do not end” button B42. The “end” button B41 is a button for instructing the ultrasound diagnostic apparatus 20 being operated to end the current examination and start a new examination. The “do not end” button B42 is a button for instructing the ultrasound diagnostic apparatus 20 being operated to continue the current examination.

Then, the hardware processor 27 determines whether or not there is an instruction to end the current examination by the user's operation through the operation inputter 23 (step S51).

When there is an instruction to end the current examination (step S51; YES), that is, when the “end” button B41 on the warning screen 263 is pressed, the hardware processor 27 performs processing of steps S45 to S47.

When there is no instruction to end the current examination in step S51 (step S51; NO), that is, when the “do not end” button B42 on the warning screen 263 is pressed, the hardware processor 27 performs processing of steps S48 and S49.

When the electronic medical record terminal 10 as a transmission source is not set as an “electronic medical record terminal as an acquisition destination” in step S43 (step S43; NO), the hardware processor 27 performs processing of steps S48 and S49.

After step S47 or step S49, the third subject information setting process ends.

As described above, according to the fifth embodiment, what to do when another subject information is received during the examination in the ultrasound diagnostic apparatus 20 can be set in advance. Specifically, it is possible to set whether the newly received subject information is to be examined, or whether the previous subject information setting is to be continued, or whether the user makes a determination when such a situation occurs.

In the fifth embodiment, the system is the same as that of the first embodiment. However, as in the second embodiment or the modification example of the second embodiment, each ultrasound diagnostic apparatus 20 may determine whether or not the electronic medical record terminal 10 as a transmission source is set as an “electronic medical record terminal as an acquisition destination” based on the information regarding the setting table managed by the server 30.

The description in each of the embodiments and the modification examples is an example of the medical examination apparatus and the medical examination system according to the present invention, and the invention is not limited thereto. The detailed configuration and detailed operation of each unit of each apparatus can be appropriately changed without departing from the spirit of the present invention. Characteristic processes in the respective embodiments and modification examples may be combined.

For example, also in the third and fourth embodiments, when the second subject information setting process (refer to FIG. 8) illustrated in the second embodiment is executed, the hardware processor 27 of the ultrasound diagnostic apparatus 20 may acquire a determination result based on the setting table from the server 30 and determine whether or not the electronic medical record terminal 10 as a transmission source is an “electronic medical record terminal as an acquisition destination” based on the determination result, as in the modification example of the second embodiment.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical examination apparatus, comprising:
an examination data generator that generates examination data by examining a subject;
a communicator that is able to communicate with a plurality of information processing apparatuses; and
a hardware processor that sets an information processing apparatus as an acquisition destination among the plurality of information processing apparatuses and that, when subject information regarding a subject and apparatus identification information for identifying any one of the plurality of information processing apparatuses are received from the information processing apparatus through the communicator, determines whether or not the information processing apparatus as a transmission source of the received subject information and apparatus identification information is the information processing apparatus as the acquisition destination based on the received apparatus identification information, and that sets the received subject information as subject information regarding a subject to be examined by the examination data generator when it is determined that the information processing apparatus as the transmission source is the information processing apparatus as the acquisition destination.

2. The medical examination apparatus according to claim 1, further comprising:
a storage that stores a setting table indicating an association as to whether or not each of the plurality of information processing apparatuses is the information processing apparatus as the acquisition destination,
wherein the hardware processor determines whether or not the information processing apparatus as the transmission source is the information processing apparatus as the acquisition destination with reference to the setting table stored in the storage.

3. The medical examination apparatus according to claim 1,
wherein the communicator is able to communicate with a server including a storage that stores a setting table in which each of a plurality of medical examination apparatuses is associated with each of the plurality of information processing apparatuses, and
the hardware processor acquires the setting table from the server through the communicator, and determines whether or not the information processing apparatus as the transmission source is the information processing apparatus as the acquisition destination by determining whether or not a medical examination apparatus associated with the information processing apparatus as the transmission source in the setting table is the medical examination apparatus.

4. The medical examination apparatus according to claim 1,
wherein the communicator is able to communicate with a server including a storage that stores a setting table in which each of a plurality of medical examination apparatuses is associated with each of the plurality of information processing apparatuses, and
the hardware processor transmits apparatus identification information of the information processing apparatus as the transmission source to the server through the communicator, acquires, from the server, a determination result indicating whether or not a medical examination apparatus associated with the information processing apparatus as the transmission source in the setting table is the medical examination apparatus, and determines whether or not the information processing apparatus as the transmission source is the information processing apparatus as the acquisition destination based on the determination result.

5. The medical examination apparatus according to claim 3,
wherein the hardware processor sets the information processing apparatus as the acquisition destination by changing the setting table.

6. The medical examination apparatus according to claim 5,
wherein the hardware processor is able to transition to an acquisition destination change mode,
when subject information and apparatus identification information for identifying any one of the information processing apparatuses are received from the information processing apparatus through the communicator during the acquisition destination change mode, the hardware processor sets the information processing apparatus as the information processing apparatus as the acquisition destination, and
the hardware processor ends the acquisition destination change mode when subject information is received from any one of the information processing apparatuses through the communicator during the acquisition destination change mode.

7. The medical examination apparatus according to claim 6,
wherein the hardware processor determines whether or not two or more information processing apparatuses associated with the same medical examination apparatus are present in the setting table, and
when it is determined that there are two or more information processing apparatuses associated with the same medical examination apparatus, the hardware processor changes the setting table so that any one of the two or more information processing apparatuses and the medical examination apparatus associated with the two or more information processing apparatuses are associated each other.

8. The medical examination apparatus according to claim 7,
wherein the hardware processor changes the setting table so that an information processing apparatus whose correspondence relationship with the same medical examination apparatus is set last, among the two or more information processing apparatuses, and the medical examination apparatus associated with the two or more information processing apparatuses are associated each other.

9. The medical examination apparatus according to claim 8,
wherein the hardware processor notifies a user of a determination result when it is determined that there are two or more information processing apparatuses associated with the same medical examination apparatus,
the hardware processor includes a selector that causes a user to select whether or not to give priority to a setting of an information processing apparatus whose correspondence relationship with the same medical examination apparatus is set last among the two or more information processing apparatuses, and
when giving priority to the setting of the information processing apparatus whose correspondence relationship with the same medical examination apparatus is set last is selected by the selector, the hardware processor changes the setting table so that the information processing apparatus whose correspondence relationship with the same medical examination apparatus is set last is associated with the medical examination apparatus associated with the two or more information processing apparatuses.

10. The medical examination apparatus according to claim 1,
wherein the subject information is a subject identification for identifying a subject.

11. A medical examination system, comprising:
a plurality of information processing apparatuses that input and display subject information regarding a subject;
a plurality of medical examination apparatuses each including an examination data generator that generates examination data by examining a subject; and
a server that is able to communicate with the plurality of medical examination apparatuses,
wherein each of the plurality of information processing apparatuses includes a transmitter that transmits subject information displayed on the information processing apparatus and apparatus identification information for identifying the information processing apparatus to each of the plurality of medical examination apparatuses,
each of the plurality of medical examination apparatuses includes:
a communicator that is able to communicate with the plurality of information processing apparatuses; and
a first hardware processor that sets an information processing apparatus as an acquisition destination among the plurality of information processing apparatuses and that, when subject information regarding a subject and apparatus identification information for identifying any one of the plurality of information processing apparatuses are received from the information processing apparatus through the communicator, determines whether or not the information processing apparatus as a transmission source of the received subject information and apparatus identification information is the information processing apparatus as the acquisition destination based on the received apparatus identification information, and that sets the received subject information as subject information regarding a subject to be examined by the examination data generator when it is determined that the information processing apparatus as the transmission source is the information processing apparatus as the acquisition destination,
the server includes a storage that stores a setting table in which each of the plurality of medical examination apparatuses is associated with each of the plurality of information processing apparatuses, and
the first hardware processor determines whether or not the information processing apparatus as the transmission source is the information processing apparatus as the acquisition destination based on information regarding the setting table acquired from the server.

12. The medical examination system according to claim 11,
wherein the server includes a second hardware processor that, when a request for transmission of the setting table is received from any one of the plurality of medical examination apparatuses, transmits the setting table to the requesting medical examination apparatus, and
the first hardware processor of the requesting medical examination apparatus acquires the setting table from the server through the communicator, and determines whether or not the information processing apparatus as the transmission source is the information processing apparatus as the acquisition destination by determining whether or not a medical examination apparatus associated with the information processing apparatus as the transmission source in the setting table is the requesting medical examination apparatus.

13. The medical examination system according to claim 11,
wherein the server includes a second hardware processor that, when the apparatus identification information of the information processing apparatus as the transmission source is received from any one of the plurality of medical examination apparatuses, determines whether or not a medical examination apparatus associated with the information processing apparatus as the transmission source is a requesting medical examination apparatus, which has transmitted the apparatus identification information to the server, based on the received apparatus identification information with reference to the setting table and transmits a result of the determination to the requesting medical examination apparatus, and
the first hardware processor of the requesting medical examination apparatus transmits the apparatus identification information of the information processing apparatus as the transmission source to the server through the communicator, acquires the determination result from the server, and determines whether or not the information processing apparatus as the transmission source is the information processing apparatus as the acquisition destination based on the determination result.

14. The medical examination system according to claim 11,
wherein the server includes:
a receiver that receives an instruction to change the setting table; and
a second hardware processor that changes a correspondence relationship between each of the plurality of medical examination apparatuses and each of the plurality of information processing apparatuses in the setting table when an instruction to change the setting table is received by the receiver.

15. The medical examination system according to claim 11,
wherein the subject information is a subject ID for identifying a subject.
